(19) United States
(12) Patent Application Publication
(10) Pub. No.: US 2021/0386379 A1
(43) Pub. Date: Dec. 16, 2021

(54) ELECTRODE COMPRISING A CONDUCTIVE ACRYLATE BASED PRESSURE SENSITIVE ADHESIVE

(71) Applicant: Henkel AG & Co. KGaA, Duesseldorf (DE)

(72) Inventors: Carla Negele, Neuss (DE); Inge van der Meulen, Eersel (NL); Stijn Gillissen, Hasselt (BE); Tobias Roschek, Solingen (DE); Frank Goethel, Chemnitz (DE); Alissa Besler, Duesseldorf (DE)

(21) Appl. No.: 17/446,484

(22) Filed: Aug. 31, 2021

Related U.S. Application Data
(63) Continuation of application No. PCT/EP2020/055387, filed on Mar. 2, 2020.

(30) Foreign Application Priority Data
Mar. 7, 2019 (EP) 19161202.7

(51) Int. Cl. A61B 5/00; A61B 5/265; A61B 5/266; A61B 5/268; C09J 9/02; C09J 133/14

(52) U.S. Cl. CPC A61B 5/68335; A61B 5/265; A61B 5/266; A61B 5/268; A61B 5/28; C09J 133/14; A61B 2562/0215; A61B 2562/0217; C09J 9/02

(57) ABSTRACT

The present invention relates to an electrode comprising a conductive pressure sensitive adhesive layer and a conductive layer. Furthermore, the invention refers to a method of manufacturing the electrode and to the use of the electrode for monitoring biosignals.

### TECHNICAL FIELD OF THE INVENTION

The present invention relates to an electrode comprising a conductive pressure sensitive adhesive layer and a conductive layer. Furthermore, the invention refers to a method of manufacturing the electrode and to the use of the electrode for monitoring biosignals.

### BACKGROUND OF THE INVENTION

Various kind of electrodes are used to measure biosignals such as electrocardiography (ECG), electroencephalography (EEG) and electromyography (EMG).

For example, currently used ECG electrodes are attached to the skin via gel, which acts as an electrolyte and transfers the body signal to the electrode. However, they dry out over time and cannot be used for prolongated measurements. In most of the cases, they are not recommended to be used longer than 24 h. In addition, they can only be stored for a relatively short period, commonly only one month after opening, and furthermore, they need a special packaging preventing them from drying out.

Especially currently used gel electrodes have high salt concentrations, which are needed for low impedances and good signal quality. However, the high salt concentrations cause skin irritation in many cases. Furthermore, these electrodes require a relatively high quantity of water. The high water content is one reason why these electrodes tend to dry out, and therefore, cannot be used for long-term measurements (in particular for more than three days), because the signal quality decreases with decreasing water content. Current gel electrodes are attached to the skin with a ring of a pressure sensitive skin adhesive surrounding the inner gel.

There are also tab electrodes currently on the market, which are attached to the skin via a gel-type adhesive. These electrodes do not need an additional skin adhesive, since the gel itself is adhering to the skin. However, these electrodes also comprise a salt and water, and can dry out over time and are therefore not suitable for prolongated measurements. The cohesion of the adhesive is often poor in these electrodes, leading to cohesive failure upon removal of the electrode.

Alternatively, a pressure sensitive adhesive comprising conductive fillers, such as carbon black can be used in the electrodes to measure biosignals. The drawback in this kind of electrodes is that a high carbon black concentration is needed, which leads to a loss in adhesion. Furthermore, the signal quality in this kind of electrodes is poor due to lacking ionic conductivity.

In another electrode solution, the electrode comprises adhesives comprising the combination of carbon black and a salt. An electrophoretic alignment of conductive fillers is required in order to obtain sufficient impedances in this solution. However, this electrophoretic activation step makes the electrode production expensive and complicated.

Therefore, there is a need for electrodes to measure biosignals, which can be used for a week without loss of signal or adhesion, which do not dry out, and sensitize or irritate the skin.

The inventors of the present invention have surprisingly found that one or more of the above-described disadvantages can be overcome by the specific electrode of the present invention comprising a conductive pressure sensitive adhesive layer, in the following referred to as adhesive layer as well, which comprises at least one acrylic polymer, which is obtained by polymerizing (meth)acrylic monomers, optionally with vinyl monomers, wherein at least 10 wt.-% of the (meth)acrylic monomers contain at least one —OH group, whereby wt.-% is based on the total weight of the acrylic polymer and at least one ionic liquid. The electrodes of the present invention not only do not dry out and can be used in long-term measurements without irritation of the skin but can also be manufactured more easily. Since no extra hydrogel is needed, the electrode can be printed at one manufacturer in a rather simple process. Due to the fact that the present electrodes do not require a gel/hydrogel, the shelf-life of the electrode is improved and less demanding packaging material is required.

### BRIEF DESCRIPTION OF THE DRAWINGS

FIG. 1a, 1b, 1c, 1d, 1e and if are cross-sectional views of electrodes according to preferred embodiments of the present invention. The following layers are shown: conductive pressure sensitive adhesive layer (10), conductive layer made of carbon (20), flexible substrate (30), conductive layer made of Ag/AgCl (40), metal layer (50), conductive layer made of Ag (60), release liner (70), conductive element (80) made of a flexible substrate (30) covered with at least one conductive layer ((20), (40), or (60)) in contact with the pressure sensitive adhesive layer (10)).

FIG. 2a, 2b, 2c, 2d, and 2e are top views illustrating preferred embodiments of conductive pressure sensitive adhesive layer (10) patterns on conductive element (80).

FIG. 3 is a graph showing impedance spectra recorded from Examples 1a-d and Comparative Example 1.

FIG. 4 shows ECG spectra recorded from Example 1c and Comparative Example 1.

FIG. 5 is a graph showing impedance spectra of compositions according to Example 1 (solid line) and 2 (dotted line) on Ag/AgCl electrodes.

FIG. 6 is a graph showing defibrillation overload recovery test curves of Examples 2-4.

FIG. 7 is a graph showing defibrillation overload recovery discharge curves according to ANSI/AAMI EC12:2000/(R)2015 for an electrode pair with electrode adhesive according to Example 2.

FIG. 8 is a graph showing defibrillation overload recovery discharge curves according to ANSI/AAMI EC12:2000/(R)2015 for an electrode pair with electrode adhesive according to Example 1.

FIG. 9 is a graph showing a voltage increase over time during current bias for electrode samples with different adhesive compositions (Examples 1 and 2).

FIG. 10 is a graph showing a voltage increase during long time current bias (200 nA) for electrode samples having an electrode adhesive (Example 1).

FIG. 11 is a graph showing a voltage increase during long time current bias (2 μA) for an electrode sample having an electrode adhesive (Example 1).

FIG. 12 is a graph of output voltage as a function of time showing an offset instability and internal noise measurement for an electrode sample having an electrode adhesive according to the present invention (Example 1).

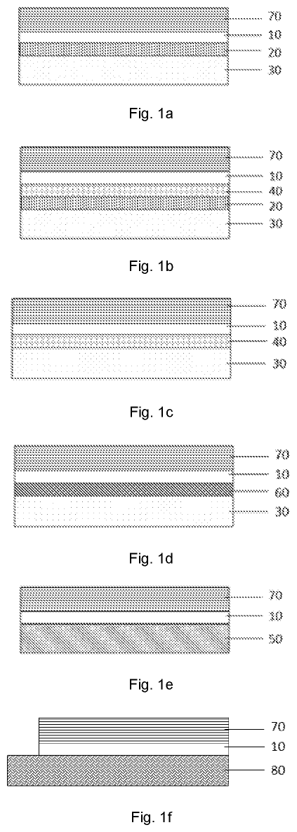

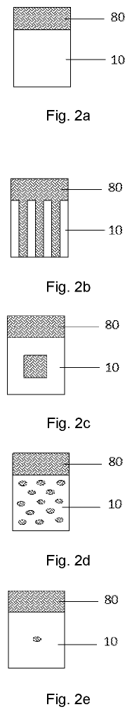

### SUMMARY OF THE INVENTION

In a first aspect the present invention refers to an electrode, comprising or consisting of
(A) a conductive pressure sensitive adhesive layer, which comprises or consists of
(A1) at least one (meth)acrylic polymer, which is obtained by polymerizing of (meth)acrylic monomers, optionally with vinyl monomers, wherein at least 10 wt.-% of the (meth)acrylic monomers contain at least one —OH group, whereby wt.-% is based on the total weight of the acrylic polymer;
(A2) at least one ionic liquid;
(A3) optionally at least one ionic conductivity promoter;
(A4) optionally at least one electrically conductive particle;
(A5) optionally at least one polyol; and
(A6) optionally at least one solvent;
(B) a conductive layer, which is in contact with the conductive pressure sensitive adhesive layer;
(C) optionally a substrate, which is in contact with the conductive layer; and
(D) optionally a release liner, which is in contact with the conductive pressure sensitive adhesive layer.

In a second aspect, the present invention pertains to a method of manufacturing an electrode according to the present invention, comprising or consisting of the steps:
(i) optionally providing a substrate upon which on one side a conductive layer is applied via flat-bed screen printing, rotary screen printing, flexo-printing, gravure printing, pad printing, inkjet printing, LIFT printing, vacuum based deposition methods, like CVC, PVD, and ALD, spray coating, dip coating or plating;
(ii) applying the conductive pressure sensitive adhesive layer upon the conductive layer via coating, laminating, spraying or printing; and
(iii) optionally applying a release liner upon the side of the conductive pressure sensitive adhesive layer.

In a final aspect, the present invention relates to the use of the electrode according to the present invention for monitoring biosignals, preferably ECG, EEG, EMG or bioimpedance.

### DETAILED DESCRIPTION OF THE INVENTION

In the following passages, the present invention is described in more detail. Each described embodiment may be combined with any other aspect or embodiment unless clearly indicated to the contrary. In particular, any feature indicated as being preferred or advantageous may be combined with any other feature or features indicated as being preferred or advantageous.

In the context of the present invention, the terms used are to be construed in accordance with the following definitions, unless a context dictates otherwise.

The term “essentially free of” means a concentration of less than 0.1 wt.-%, preferably less than 0.01 wt.-%, more preferably less than 0.001 wt.-%, more preferably less than 0.0001 wt.-%, in particular free of the compound or substance, if it is not explicitly stated otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical end points includes all numbers and fractions subsumed within the respective ranges, as well as the recited end points.

All percentages, parts, proportions and then like mentioned herein are based on weight unless otherwise indicated.

When an amount, a concentration or other values or parameters is/are expressed in form of a range, a preferable range, or a preferable upper limit value and a preferable lower limit value, it should be understood as that any ranges obtained by combining any upper limit or preferable value with any lower limit or preferable value are specifically disclosed, without considering whether the obtained ranges are clearly mentioned in the context.

All references cited in the present specification are hereby incorporated by reference in their entirety.

Unless otherwise defined, all terms used in disclosing the invention, including technical and scientific terms, have the meaning as commonly understood by one of the ordinary skilled in the art. By means of further guidance, term definitions are included to better appreciate the teaching of the present invention.

The present invention pertains to an electrode, which does not require a gel or hydrogel, therefore the term “dry electrode” is employed for the electrode according to the present invention as well.

The electrode comprises a conductive pressure sensitive adhesive layer, which comprises or consist of (A1) at least one acrylic polymer, which is obtained by polymerizing (meth)acrylic monomers, optionally with vinyl monomers, wherein at least 10 wt.-% of the (meth)acrylic monomers contain at least one —OH group, whereby wt.-% is based on the total weight of the acrylic polymer and at least one ionic liquid.

The adhesive suitable in the present invention is a conductive pressure sensitive adhesive (PSA), in particular ionically conductive, with low impedance and good skin compatibility. The adhesive is present in the electrode in the form of a layer, which offers a solution for a long-term monitoring of biosignals by acting as a functional contact between electrode and skin. In contrast to gel-type electrodes currently in the market, it cannot dry out and it does not lead to skin irritation. Furthermore, the impedance of the PSA according to the present invention is very low without any addition of water.

The conductive pressure sensitive adhesive according to the present invention is based on a polar solvent-based acrylic pressure sensitive adhesive with high breathability and a non-toxic, non-irritating ionic liquid leading to ionic conductivity.

In one embodiment in the adhesive layer the (meth)acrylic monomers containing at least one —OH group are present in at least 15 wt.-%, preferably at least 20 wt.-%, more preferably at least 25 wt.-%, most preferably at least 30 wt.-% and/or at most 65 wt.-%, preferably at most 60 wt.-%, more preferably at most 55 wt.-%, most preferably at most 50 wt.-%, based on the total weight of the acrylic polymer. When the content of the (meth)acrylic monomers comprising at least one —OH group in said (meth)acrylic polymer is more than 65% by weight of the total weight of the (meth)acrylate polymer, the higher OH-group content may negatively affect the adhesion properties.

In another embodiment in the adhesive layer the (meth)acrylic monomers are selected from methyl (meth)acrylate, hydroxyethyl acrylate, hydroxypropyl acrylate, hydroxybutyl acrylate, butyl acrylate, ethylhexylacrylate, acrylic acid, C2-C18 alkyl (meth)acrylate, (meth)acrylamide; cyclohexyl (meth)acrylate, glycidyl (meth)acrylate, and benzyl (meth)acrylate.

In a further embodiment in the adhesive layer the vinyl monomer is selected from vinyl acetate, N-vinyl caprolactam, acrylonitrile, and vinyl ether.

In another embodiment in the adhesive layer the (meth)acrylic monomers are selected from a mixture of hydroxyethyl acrylate and at least one of methyl (meth)acrylate, butyl acrylate, ethylhexylacrylate or are selected from a mixture of hydroxyethyl acrylate and at least one of methyl (meth)acrylate, butyl acrylate, and ethylhexylacrylate.

Suitable commercially available (meth)acrylic polymers for use in the present invention include, but are not limited to LOCTITE DURO-TAK 222A, LOCTITE DURO-TAK 87-202A; LOCTITE DURO-TAK 87-402A; LOCTITE DURO-TAK 73-626A from Henkel.

The applicant has found out that a pressure sensitive adhesive based on at least one acrylic polymer, which is obtained by polymerizing (meth)acrylic monomers, optionally with vinyl monomers, wherein at least 10 wt.-% of the (meth)acrylic monomers contain at least one —OH group, whereby wt.-% is based on the total weight of the acrylic polymer, provides good impedance and electrodes do not dry out and they can be used for longer period measurement (the higher OH content increases the water vapor transmission rate of the polymer, which contributes to increased breathability and longer wear times).

In one embodiment in the adhesive layer the polyol is selected from polyether polyol, preferably from polyethylene glycol, polypropylene glycol, polytetramethylene glycol, and more preferably polyethylene glycol having weight averaged molecular weight from 300 to 1000 g/mol or from 350 to 750 g/mol or from 380 to 420 g/mol, wherein the molecular weight is measured by gel permeation chromatography according to DIN 55672-1:2007-08 with THF as the eluent. The adhesive layer according to the present invention may further comprise a polyether polyol. Preferably, the polyether polyol is selected from polyethylene glycol (PEG), polypropylene glycol (PPG), polytetramethylene glycol (PTMG) and mixture thereof. The applicant has found out that addition of polyether polyol is an exceptionally good host for ionic conductivity due to the open and flexible molecule chains, and therefore, has a positive impact on the impedance. The applicant has found out that already a small quantity of polyether polyol has a positive impact, which is beneficial regarding the skin compatibility of the composition. Suitable commercially available polyether polyols for use in the present invention include, but not limited to Kollisolv PEG 400 from BASF.

In a further embodiment in the adhesive layer the polyol is present in 0.1 to 50 wt.-%, or 0.5 to 20 wt.-%, based on the total weight of the adhesive layer.

In another embodiment in the adhesive layer the solvent is selected from the group consisting of water, ethyl acetate, butyl acetate, butyl diglycol, 2-butoxyethanol, ethylene glycol, diethylene glycol, propylene glycol, dipropylene glycol, methanol, isopropanol, butanol, dibasic esters, hexane, heptane, 2,4-pentadione, toluene, xylene, benzene, hexane, heptane, methyl ethyl ketone, methyl isobutyl ketone, diethylether and mixtures thereof, preferably said solvent is selected from the group consisting of ethyl acetate, butyl acetate, ethylene glycol, propylene glycol and mixtures thereof.

In a further embodiment in the adhesive layer the solvent is present in 0.001 to 10 wt.-%, preferably 0.001 to 5 wt.-%, more preferably 0.01 to 1 wt.-%, based on the total weight of the conductive pressure sensitive adhesive layer (A).

Most preferably, the adhesive layer is essentially free of a solvent, preferably the solvent as defined above.

In an embodiment in the adhesive layer the (meth)acrylic polymer (A1) is present in 10 to 99 wt.-%, or 15 to 97 wt.-%, or 50 to 95 wt.-%, based on the total weight of the conductive pressure sensitive adhesive layer (A). Lower (meth)acrylate polymer quantities than 10 wt.-% may lead to poor adhesion properties and are not beneficial to film forming properties.

An adhesive layer according to the present invention comprises an ionic liquid, preferably a non-toxic, non-irritating ionic liquid leading to ionic conductivity.

In another embodiment in the adhesive layer the ionic liquid (A2) is selected from the group consisting of imidazolium acetates, imidazolium sulfonates, imidazolium chlorides, imidazolium sulphates, imidazolium phosphates, imidazolium thiocyanates, imidazolium dicyanamides, imidazolium benzoates, imidazolium triflates, choline triflates, choline saccharinate, choline sulfamates, pyridinium acetates, pyridinium sulfonates, pyridinium chlorides, pyridinium sulphates, pyridinium phosphates, pyridinium thiocyanates, pyridinium dicyanamides, pyridinium benzoates, pyridinium triflates, pyrrolidinium acetates, pyrrolidinium sulfonates, pyrrolidinium chlorides, pyrrolidinium sulphates, pyrrolidinium phosphates, pyrrolidinium thiocyanates, pyrrolidinium dicyanamides, pyrrolidinium benzoates, pyrrolidinium triflates, phosphonium acetates, phosphonium sulfonates, phosphonium chlorides, phosphonium sulphates, phosphonium phosphates, phosphonium thiocyanates, phosphonium dicyanamides, phosphonium benzoates, phosphonium triflates, sulfonium acetates, sulfonium sulfonates, sulfonium chlorides, sulfonium sulphates, sulfonium phosphates, sulfonium thiocyanates, sulfonium dicyanamides, sulfonium benzoates, sulfonium triflates, ammonium acetates, ammonium sulfonates, ammonium chlorides, ammonium sulphates, ammonium phosphates, ammonium thiocyanates, ammonium dicyanamides, ammonium benzoates, ammonium triflates and mixtures thereof.

In a further embodiment in the adhesive layer the ionic liquid is selected from the group consisting of 1-ethyl-3-methylimidazolium acetate, 1-ethyl-3-methylimidazolium methanesulfonate, 1-ethyl-3-methylimidazolium trifluoromethanesulfonate, 1-ethyl-3-methylimidazolium chloride, 1-ethyl-3-methylimidazolium ethyl sulphate, 1-ethyl-3-methylimidazolium diethyl phosphate, 1-ethyl-3-methylimidazolium thiocyanate, 1-ethyl-3-methylimidazolium dicyanamide, 1-ethyl-3-methylimidazolium benzoate, choline trifluoromethane sulfonate, choline saccharinate, choline acesulfamate, choline N-cyclohexylsulfamate, tris(2-hydroxyethyl)methylammonium methyl sulphate, 1-ethyl-3-methylimidazolium tetrafluoroborate, 1-allyl-3-methylimidazolium bis(trifluoromethylsulfonyl)imide, choline acetate and mixtures thereof.

Preferably, said ionic liquid is selected from the group consisting of 1-ethyl-3-methylimidazolium acetate, 1-ethyl-3-methylimidazolium methanesulfonate, 1-ethyl-3-methylimidazolium trifluoromethanesulfonate, 1-ethyl-3-methylimidazolium chloride, 1-ethyl-3-methylimidazolium ethylsulphate, 1-ethyl-3-methylimidazolium diethyl phosphate, 1-ethyl-3-methylimidazolium thiocyanate, 1-ethyl-3-methylimidazolium dicyanamide, 1-ethyl-3-methylimidazolium benzoate, choline trifluoromethanesulfonate, choline saccharinate, choline acesulfamate, choline N-cyclohexylsulfamate, tris(2-hydroxyethyl)methylammonium methylsulphate, 1-ethyl-3-methylimidazolium tetrafluoroborate, 1-allyl-3-methylimidazolium bis(trifluoromethylsulfonyl)imide, choline acetate, and mixtures thereof.

More preferably, the ionic liquid is selected from the group consisting of 1-ethyl-3-methylimidazolium benzoate, 1-ethyl-3-methylimidazolium tetrafluoroborate, 1-ethyl-3-methylimidazolium methanesulfonate, 1-ethyl-3-methylimidazolium chloride, 1-ethyl-3-methylimidazolium trifluoromethanesulfonate, choline trifluoromethanesulfonate, 1-ethyl-3-methylimidazolium acetate, choline acetate, 1-ethyl-3-methylimidazolium diethylphosphate, 1-allyl-3-methylimidazolium bis(trifluoromethylsulfonyl)imide, 1-ethyl-3-methylimidazolium ethyl sulphate, 1-ethyl-3-methylimidazolium thiocyanate, 1-ethyl-3-methylimidazolium dicyanamide, choline saccharinate, choline acesulfamate, and mixture thereof.

Above mentioned ionic liquids are preferred because they show good solubility in the (meth)acrylic polymers according to the present invention and low toxicity.

In one embodiment two or more ionic liquids are used, in this embodiment said ionic liquids are selected from the group consisting of 1-ethyl-3-methylimidazolium acetate, 1-ethyl-3-methylimidazolium methane sulfonate, 1-ethyl-3-methylimidazolium trifluoromethane sulfonate, 1-ethyl-3-methylimidazolium chloride, 1-ethyl-3-methylimidazolium ethyl sulphate, 1-ethyl-3-methylimidazolium diethylphosphate, 1-ethyl-3-methylimidazolium thiocyanate, 1-ethyl-3-methylimidazolium dicyanamide, 1-ethyl-3-methylimidazolium benzoate, choline trifluoromethanesulfonate, choline saccharinate, choline acesulfamate, choline N-cyclohexylsulfamate, tris(2-hydroxyethyl)methylammonium methyl sulphate, 1-ethyl-3-methylimidazolium tetrafluoroborate, 1-allyl-3-methylimidazolium bis(trifluoromethylsulfonyl)imide, choline acetate;

preferably two or more ionic liquids are selected from the group consisting of 1-ethyl-3-methylimidazolium benzoate, 1-ethyl-3-methylimidazolium tetrafluoroborate, 1-ethyl-3-methylimidazolium methane sulfonate, 1-ethyl-3-methylimidazolium chloride, 1-ethyl-3-methylimidazolium trifluoromethane sulfonate, choline trifluoromethane sulfonate, 1-ethyl-3-methylimidazolium acetate, choline acetate, 1-ethyl-3-methylimidazolium diethylphosphate, 1-allyl-3-methylimidazolium bis(trifluoromethylsulfonyl)imide, 1-ethyl-3-methylimidazolium ethyl sulphate, 1-ethyl-3-methylimidazolium thiocyanate, 1-ethyl-3-methylimidazolium dicyanamide, choline saccharinate, choline acesulfamate.

Suitable commercially available ionic liquids for use in the present invention include, but are not limited to Basionics ST80, Basionics Katl, Basionics BC01, Basionics VS11, Basionics VS03, and Efka IO 6785, all from BASF.

In an embodiment in the adhesive layer the ionic liquid is present in 0.5 to 50 wt.-% or in 1 to 40 wt.-% or in 4 to 25 wt.-%, based on the total weight of the conductive pressure sensitive adhesive layer.

The adhesive layer according to the present invention may further comprise an ionic conductivity promoter, preferably a non-toxic, non-irritating ionic conductivity promoter leading to additional ionic conductivity.

The ionic conductivity promoter is semi-solid or solid under room temperature and can be dissolved in the ionic liquid. It has good compatibility with the (meth)acrylate polymer according to the present invention.

The ionic conductivity promoter suitable for the present invention is selected from choline chloride, choline bitartrate, choline dihydrogen citrate, choline phosphate, choline gluconate, choline fumarate, choline carbonate, choline pyrophosphate, sodium chloride, lithium chloride, potassium chloride, calcium chloride, magnesium chloride, aluminium chloride, silver chloride, ammonium chlorides, alkylammonium chlorides, dialkylammonium chlorides, trialkylammonium chlorides, tetraalkylammonium chlorides and mixture thereof.

In an embodiment in the adhesive layer the ionic conductivity promoter is present in 0.1 to 30 wt.-% or in 0.5 to 20 wt.-% or in ito 15 wt.-%, based on the total weight of the conductive pressure sensitive adhesive layer. If the quantity of the ionic conductivity promoter is too low, the adhesive may not show any ionic conductivity and the signal may be lost, whereas too high quantity may not provide improvement in signal quality but may increase the chances of skin irritation and decrease the adhesion properties.

The adhesive layer according to the present invention may further comprise electrically conductive particles.

In another embodiment in the adhesive layer the electrically conductive particles are selected from the group consisting of metal (nano)particles, graphite (nano)particles, carbon (nano)particles, carbon nanowires, conductive polymer (nano)particles, and mixtures thereof, more preferably selected from the group consisting of silver containing particles, silver particles, copper particles, copper containing particles, silver nanowires, copper nanowires, graphite particles, carbon particles and mixtures thereof, and even more preferably selected from graphite particles, carbon particles and mixtures thereof.

Graphite particles and carbon particles are preferred due the fact that they do not cause skin irritation, but provide adequate conductivity. Suitable commercially available electrically conductive particles for use in the present invention include, but are not limited to Ensaco 250G, Timrex KS6 from Timcal, Printex XE2B from Necarbo, C-Nergy Super C65 from Imerys and Vulcan XC72R from Cabot.

An ionically conductive pressure sensitive adhesive composition according to the present invention may comprise said electrically conductive particles from 0.1 to 35% by weight of the total weight of the composition, preferably from 0.5 to 25%, and more preferably from 1 to 15%.

If the quantity of the electrically conductive particles is too low, it may lead to poor conductivity, whereas too high quantity may lead to loss of adhesion properties.

The adhesive layer according to the present invention may further comprise a solvent. Preferably, the solvent, which may be comprised in the adhesive before drying, should be evaporated during drying whereby the adhesive layer can be formed. In a preferred embodiment, the adhesive layer is essentially free of the solvent after the drying step.

Suitable solvent for use in the present invention may be selected from the group consisting of water, ethyl acetate, butyl acetate, butyl diglycol, 2-butoxyethanol, ethylene glycol, diethylene glycol, propylene glycol, dipropylene glycol, methanol, isopropanol, butanol, dibasic esters, hexane, heptane, 2,4-pentadione, toluene, xylene, benzene, hexane, heptane, methyl ethyl ketone, methyl isobutyl ketone, diethylether and mixtures thereof, preferably said solvent is selected from the group consisting of ethyl acetate, butyl acetate, ethylene glycol, propylene glycol and mixtures thereof.

Suitable commercially available solvents for use in the present invention include, but are not limited to ethyl acetate and ethylene glycol from Brenntag, butyl acetate from Shell Chemicals and propylene glycol from Lyondell.

The adhesive layer according to the present invention may comprise a solvent from 0.001 to 10 wt.-%, preferably 0.001 to 5 wt.-%, more preferably 0.01 to 1 wt.-%, based on the total weight of the conductive pressure sensitive adhesive layer (A).

Most preferably, the adhesive layer is essentially free of the solvent.

The adhesive layer according to the present invention preferably has an impedance value below 1,000,000 Ohm at 1000 Hz, preferably below 100,000 Ohm at 1000 Hz and more preferably below 40,000 Ohm at 1000 Hz, wherein said impedance is measured by connecting two electrodes coated each with 25 μm of an ionic conductive pressure sensitive adhesive having a contact area of 0.25 cm².

The adhesive layer according to the present invention, the combination of the (meth)acrylate polymer and the ionic liquid leads to a low impedance. The ionic liquid provides the ionic conductivity. However, if the ionic liquid is not miscible with the (meth)acrylate polymer, one will see poor ionic conductivity in the pressure sensitive adhesive. In the embodiment, wherein PEG is added to the composition, the additional ether groups from the PEG make the system more polar and enhance the ionic conductivity of the ionic liquid in the (meth)acrylate polymer.

An adhesive layer composition according to the present invention commonly has high breathability. Good breathability is obtained, if the water can penetrate easily through the adhesive layer. To achieve this effect, a polar polymer is required, in this occasion, the OH-functionalities support and improve the breathability.

The adhesive layer according to the present invention preferably has a breathability value of about 4600 g/m² in 24 hours. As a comparison, a standard acrylic PSA has a breathability value of about 2000 g/m² in 24 hours. The breathability is measured through a moisture vapor transmission rate (MVTR) measurement according to ASTM D1653-13.

The adhesive layer can be obtained by coating the conductive pressure sensitive adhesive on a supporting substrate (such as a film) and drying the layer in an oven at for example 120° C. for 3 minutes to remove the solvent and form a dry layer of the conductive pressure sensitive adhesive on the supporting substrate. Commonly known methods used for preparing pressure-sensitive adhesive can be employed. Examples include roll coating, gravure coating, reverse coating, roll brushing, spray coating, and air knife coating methods, immersing and curtain coating method, and extruding coating method with a die coater.

In a preferred embodiment, the adhesive layer has a thickness of 1 to 200 μm, or 10 to 50 μm; and/or has an impedance value of 10¹ to 10⁷Ω, or 10² to 10⁵Ω at 10 Hz. Wherein the adhesive layer has a surface area from 0.25 cm² to 10 cm², preferably from 1 cm² to 6 cm².

The electrode according to the present invention contains a conductive layer, preferably only one conductive layer.

In one embodiment the conductive layer is selected from a metal or metal salt layer, in particular a copper, silver, gold, aluminium, Ag/AgCl, or a carbon layer or mixtures thereof.

In another embodiment the conductive layer has a thickness of 0.1 to 500 μm, or 0.5 to 150 μm, or 1 to 25 μm, or 1 to 20 μm.

In a further embodiment the conductive layer is the only conductive layer contained in the electrode in addition to the conductive pressure sensitive adhesive.

In preferred embodiments, the electrode according to the present invention contains a substrate. In one embodiment the substrate is a flexible film, preferably selected from polyolefin films, polycarbonate films, thermoplastic polyurethane (TPU) films, silicone films, woven films, non-woven films, or paper films, in particular polyethylene films, polypropylene films, polyethylene terephthalate films or thermoplastic polyurethane films.

In another embodiment the substrate has a thickness of 10 to 500 μm, or 25 to 150 μm.

In one embodiment, the conductive layer (B) is a metal, preferably with a thickness of 10 to 500 μm, or 25 to 150 μm. Preferably, the metal is a copper, silver, gold, or aluminium layer.

In order to package the electrode and avoid that the adhesive layer sticks to the package, the electrode can contain a release liner on the surface of the adhesive layer which is later applied to the area which should be measured. All known release liners in the art are suitable, in one embodiment the release liner is selected from siliconized paper release liner or plastic release liner.

As already stated above the electrode of the present invention does not require a gel/hydrogel. Therefore, in one embodiment, the electrode is essentially free from a hydrogel, preferably does not contain more than 0.5 wt.-%, or 0.1 wt.-%, or 0.001 wt.-% of a hydrogel, or does not contain a hydrogel, based on the total weight of the electrode.

In another embodiment the electrode is essentially free from an aqueous electrolyte paste, preferably does not contain more than 0.5 wt.-%, or 0.1 wt.-%, or 0.001 wt.-% of an aqueous electrolyte paste, or does not contain an aqueous electrolyte paste, based on the total weight of the electrode.

In a further embodiment the electrode is essentially free from water, preferably does not contain more than 2 wt.-%, or 0.5 wt.-%, or 0.01 wt.-% of water, or does not contain water, based on the total weight of the electrode.

Impedance is the key parameter for the functionality of electrodes and the requirements and measurement procedures for disposable ECG electrodes are defined by ANSI/AAMI EC12:2000/(R)2015. The impedance of the electrodes at 10 Hz is required to be below 2000 Ohm on average for two electrodes attached to each other with their adhesive sides. The electrode impedance at 10 Hz is dominated by the impedance of the adhesive for a suitable conductive layer material.

In addition to the impedance requirement, a certain defibrillation overload recovery must be provided by medical ECG electrodes (measurement is done according to ANSI/AAMI EC12:2000/(R)2015). In this context, defibrillation overload recovery refers to the voltage decrease across the electrodes while a 10 μF capacitor (charged to 200V) is discharged via the sample (which consists of two electrodes attached to each other via their adhesive sides; electrode corresponds here to an adhesive on an Ag/AgCl conductive layer on a non-conductive substrate). For a successful test this has to be fulfilled 3 times in a row. The allowed voltage ranges are shown in the table 1 below, values are either maximum allowed voltages at a time or maximum allowed voltage differences within a time interval:

TABLE 1

| | |
|---|---|
| Time | Need (mV) |

| | | | |
|---|---|---|---|
| 2 | s | < | 2000 |
| 7 | s | < | 100 |
| 7-17 | s | <Δ | 11 |
| 17-27 | s | <Δ | 11 |

The defibrillation overload recovery may be influenced by the selection of the ionic liquid/salt, especially the anion of the ionic liquid/salt. Especially chloride provides fast defibrillation overload recovery times on Ag/AgCl electrodes. In principle, every chloride may be used, however, chlorides of ionic liquids (e.g. EMIM chloride or choline chloride) are preferred due to their good compatibility with the adhesive material. However, EMIM chloride in the adhesive composition may not lead to sufficient bulk conductivity to pass the impedance requirements. Surprisingly, ionic liquids with anions providing good bulk conductivity (e.g. EMIM dicyanamide) do not show a fast defibrillation overload recovery. Therefore, there is a need to find a good balance between good bulk conductivity and fast discharge properties for the ideal electrode behaviour. A combination of two or more different ionic liquids or salts in an ionically conductive PSA according to the present invention may be a solution to meet all performance requirements of electrodes.

It has been found that chloride salts provide fast discharge properties already in lower quantities (<2 wt % of the dry adhesive film according to the present invention) because electrodes with adhesives comprising chlorides have a DC resistance in the kOhm range, whereas electrodes with adhesives without chlorides have a DC resistance about 10 MOhm. Only a low DC resistivity allows the sample to discharge in a short time, and therefore, the defibrillation overload recovery requirement can be met.

The electrode of the present invention is manufactured via a method comprising or consisting of the steps:
(i) optionally providing a substrate upon which on one side a conductive layer is applied via flat-bed screen printing, rotary screen printing, flexo-printing, gravure printing, pad printing, inkjet printing, LIFT printing, vacuum based deposition methods, like CVC, PVD, and ALD, spray coating, dip coating or plating;
(ii) applying the conductive pressure sensitive adhesive layer upon the conductive layer via coating, laminating, spraying or printing; and
(iii) optionally applying a release liner upon the side of the conductive pressure sensitive adhesive layer.

In one embodiment in step (ii) the conductive pressure sensitive adhesive layer partially or fully covers the surface of the conductive layer.

Preferably, the conductive pressure sensitive adhesive layer is a printable material. Thereby, the layer (A) can be applied on only parts of the conductive layer (B) in a very easy manner. The layer application on only parts of the conductive layer may improve the breathability of the whole electrode and therefore even reduces skin irritation.

Therefore, in a preferred embodiment, the conductive pressure sensitive adhesive layer is applied on only parts of the conductive layer. It is possible to apply the conductive pressure sensitive adhesive layer on the conductive layer in different patterns. Preferably, the conductive pressure sensitive adhesive layer forms no continuous layer on the whole surface of the conductive layer.

In another embodiment after the application of the conductive pressure sensitive adhesive layer the layer is cured for is to 2 h, preferably 3 s to 10 min, preferably at 20 to 150° C., more preferably at 80 to 130° C.

In a further embodiment after the application of the conductive layer, the conductive layer is dried for is to 2 h, preferably 3 s to 15 min, preferably at 20 to 200° C., more preferably 30 to 150° C.

The electrodes according to the present invention are used for monitoring biosignals, preferably ECG, EEG, EMG or bioimpedance.

### EXAMPLES

### Materials:

DURO-TAK 222A from Henkel AG & Co. KGaA

1-ethyl-3-methylimidazolium trifluoromethanesulfonate from Proionic

1-ethyl-3-methylimidazolium dicyanamide from BASF

1-ethyl-3-methylimidazolium chloride from BASF

### Example 1 and Comparative Example 1

### Conductive PSA Preparation:

5 g LOCTITE Duro-TAK 222A (solid content: 41%) and 0.171 g of 1-ethyl-3-methylimidazolium trifluoromethanesulfonate and 0.057 g of 1-ethyl-3-methylimidazolium chloride were mixed in a conditioning mixer for 3 minutes at 2000 rpm. The mixture was coated onto a release liner and dried at room temperature for 30 min yielding PSA films with a thickness of 20 μm. Subsequently the drawdown was cured at 120° C. for 3 min and covered with another release liner.

### ECG Electrode Preparation Containing Conductive PSA:

Various conductive layers were covered with the conductive PSA and adhered together such that the connected area was 3.1 cm². The electrode pair was connected with alligator clips and the impedance of the capacitor was measured.

Comparative Example 1: 3M Red Dot 2330 Resting ECG Electrode

Example 1a: Conductive PSA on carbon layer (thickness: 14 μm); carbon layer prepared with LOCTITE ECI 7005 E&C on TPU substrate

Example 1b: Conductive PSA on Ag layer (thickness: 5 μm); Ag layer prepared with LOCTITE ECI 1010 E&C on TPU substrate

Example 1c: Conductive PSA on Ag/AgCl layer (thickness: 12 μm); Ag/AgCl layer prepared with LOCTITE EDAG 6038E SS E&C on TPU substrate

Example 1d: Conductive PSA on conductive element (thickness: 10 μm) taken from comparative ex. 1

FIG. 3 shows that according to the present invention ECG electrodes comprising a conductive PSA (Examples 1a-d) have similar impedance spectra compared with a commercial Resting ECG electrode (comparative example 1). In all examples, the impedance at 10 Hz is below 2000 Ohm, fulfilling performance requirements according to ANSI/AAMI EC12:2000.

FIG. 3 shows that Examples 1a-d lead to impedance spectra comparable with the commercial conductive elements. The commercial element was used by removing the hydrogel from a commercial tab electrode. The obtained conductive element was coated with a conductive adhesive according to the present invention and measured in a capacitor setup as comparative sample.

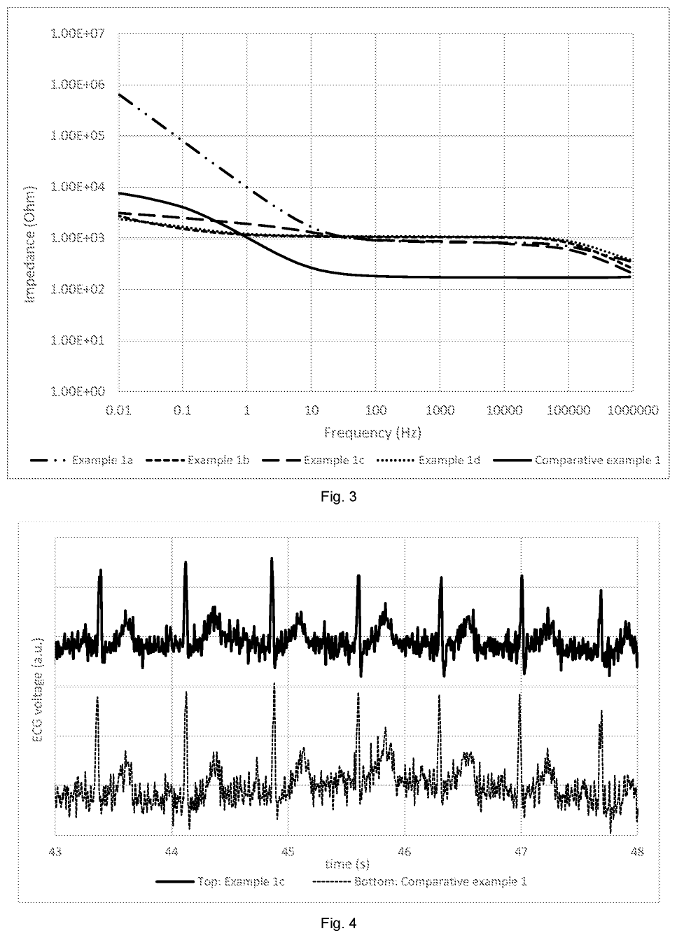

FIG. 4 illustrates the recorded ECG spectra. ECG signals were recorded using three electrodes (working-, counter- and reference electrode) placed at the inner side of the human forearms (two on the left arm, one on the right arm) and the derivation was measured between left and right arm. The monitoring took place while resting the arms. In all cases good ECG signals could be obtained.

### Example 2

5 g LOCTITE Duro-TAK 222A (solid content: 41%) and 0.228 g of 1-ethyl-3-methylimidazolium trifluoromethanesulfonate were mixed in a conditioning mixer for 3 minutes at 2000 rpm. The mixture was coated onto a release liner and dried at room temperature for 30 min yielding PSA films with a thickness of 20 μm. Subsequently the drawdown was cured at 120° C. for 3 min and covered with another release liner.

### Example 3

5 g LOCTITE Duro-TAK 222A (solid content: 41%) and 0.228 g of 1-ethyl-3-methylimidazolium dicyanamide were mixed in a conditioning mixer for 3 minutes at 2000 rpm. The mixture was coated onto a release liner and dried at room temperature for 30 min yielding PSA films with a thickness of 20 μm. Subsequently the drawdown was cured at 120° C. for 3 min and covered with another release liner.

### Example 4

5 g LOCTITE Duro-TAK 222A (solid content: 41%) and 0.228 g of 1-ethyl-3-methylimidazolium chloride were mixed in a conditioning mixer for 3 minutes at 2000 rpm. The mixture was coated onto a release liner and dried at room temperature for 30 min yielding PSA films with a thickness of 20 μm. Subsequently the drawdown was cured at 120° C. for 3 min and covered with another release liner.

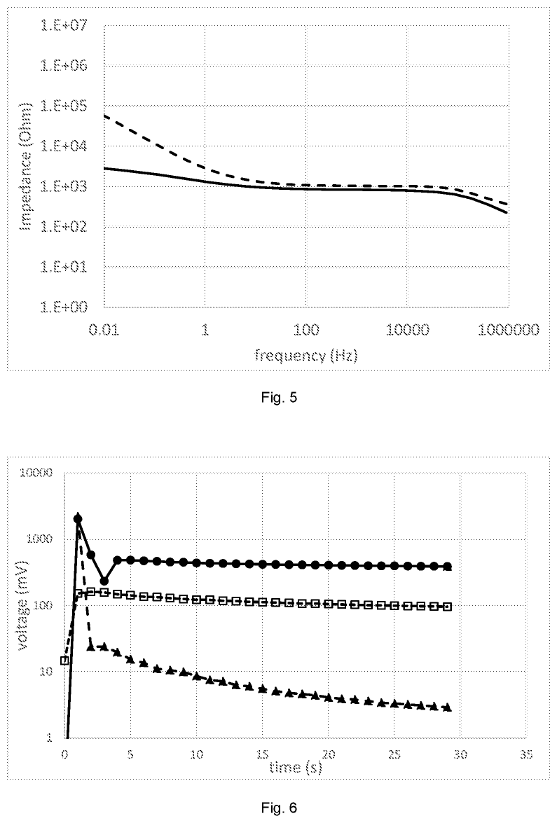

FIG. 5 illustrates impedance curves of electrodes with Ag/AgCl conductive layers and adhesive compositions according to Example 1 (solid line) and 2 (dotted line). The major difference is the increase at low frequencies indicating a lower interface (DC) conductivity for Example 1.

FIG. 5 illustrates that impedance spectra of electrodes with Ag/AgCl conductive layer without chloride in the adhesive show a strong capacitive increase at low frequencies corresponding to the existence of a blocking electrode and therefore a high DC resistance since (almost) no charge transfer across the electrode/adhesive interface occurs. In contrast to that, electrodes with adhesives comprising chlorides allow reactions between the Ag/AgCl conductive layer and the electrode adhesive leading to charge transfer (at suitable low voltages) and therefore low DC resistance which enables a fast discharge during DOR experiments.

Defibrillation overload recovery was tested for Examples 2-4. In this test voltage over time during discharge for different electrode adhesive compositions (Example 2 (circles), Example 3 (squares), Example 4 (triangles)) was measured (FIG. 6). FIG. 6 illustrates the voltage across the electrodes during discharge. For Examples 2 and 3 the voltage is constantly above 100 mV indicating that no sufficient discharge takes place (condition 2 of table 2 is missed_<100 mV after 7 s) whereas sample 4 easily passes the test requirements.

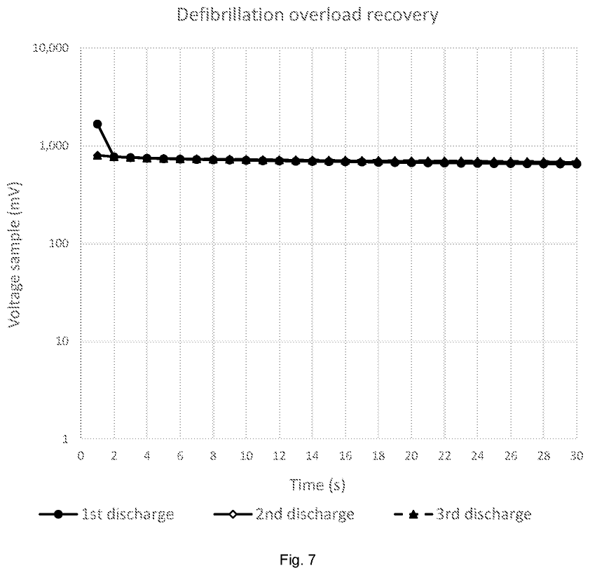

FIG. 7 illustrates three consecutive defibrillation overload recovery discharge curves according to ANSI/AAMI EC12:2000/(R)2015 for an electrode pair with electrode adhesive according to the example 2. An overview of test conditions for an electrode pair with electrode adhesive according to the example 2 is illustrated in Table 2 below. Three out of four requirements were not met showing the need for an adhesive that allows a faster discharge.

TABLE 2

| | | | | Example 2 | | |
|---|---|---|---|---|---|---|
| Time | | Need (mV) | | 1st discharge | 2nd discharge | 3^{rd} discharge |
| 2 | s | < | 2000 | 759 | 765 | 765 |
| 7 | s | < | 100 | 718 | 730 | 734 |
| 7-17 | s | <Δ | 11 | 39 | 29 | 25 |
| 17-27 | s | <Δ | 11 | 25 | 21 | 16 |

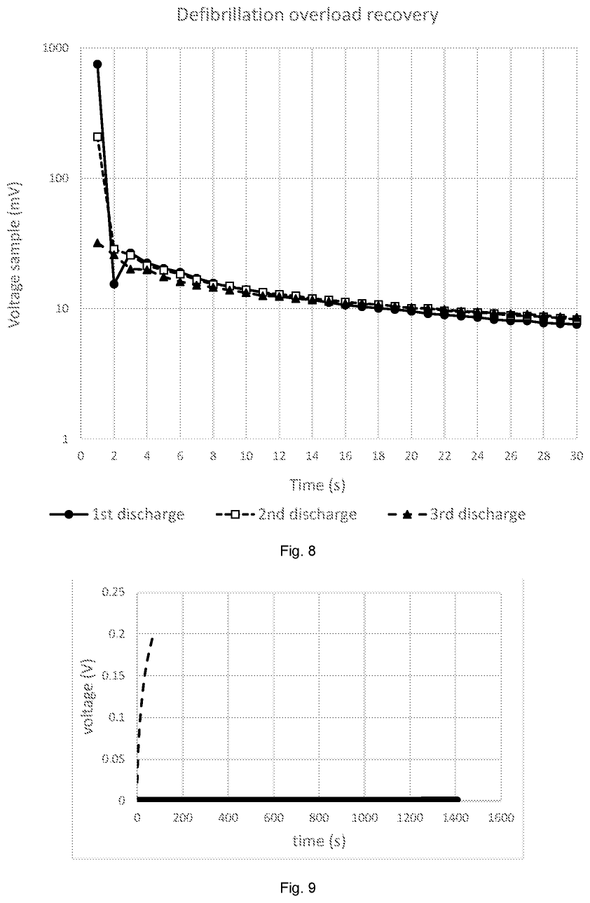

FIG. 8 illustrates three consecutive defibrillation overload recovery discharge curves according to ANSI/AAMI EC12:2000/(R)2015 for an electrode pair with electrode adhesive according to the Example 1. An overview of test conditions for an electrode pair with electrode adhesive according to the Example 1 is illustrated in table 3 below.

TABLE 3

| | | | | Example 2 | | |
|---|---|---|---|---|---|---|
| Time | | Need (mV) | | 1^{st} discharge | 2^{nd} discharge | 3^{rd} discharge |
| 2 | s | < | 2000 | 26.9 | 25.9 | 20.2 |
| 7 | s | < | 100 | 15.8 | 15.4 | 14.6 |
| 7-17 | s | <Δ | 11 | 5.7 | 4.6 | 4 |
| 17-27 | s | <Δ | 11 | 2.3 | 2.1 | 1.7 |

Here all requirements are met showing the benefit of adding a DC conductivity enabling ionic liquid like a chloride.

ANSI/AAMI EC12:2000/(R)2015 describes that the use time of an electrode is limited to the time a sample (two electrodes attached to each other via their adhesive sides) can be biased with 200 nA current at a resulting voltage <100 mV. A DC offset >100 mV should not be measured. This value correlates to the starting points of the current bias curves.

FIG. 9 illustrates a voltage increase during current bias for electrode samples with different adhesive compositions according to the present invention: Example 1—solid line and Example 2—dotted line. Example 1 corresponds to a sample with DC conductivity. The voltage is defined by Ohm's law. This voltage can be maintained for a long time. Since DC conductivity corresponds to a reversible electrochemical reaction at the interface, the voltage will stay relatively constant as long as reactants are available at the interface. In case of example 2 there was no significant DC conductivity across the interface. Therefore, the voltage corresponds to a charging of the interface capacitance is therefore steeply increasing with time.

Electrodes that provide a DC conductivity also show longer bias current tolerance and lower DC offset values. Preferably, electrode adhesives show both DC conductivity and low impedance.

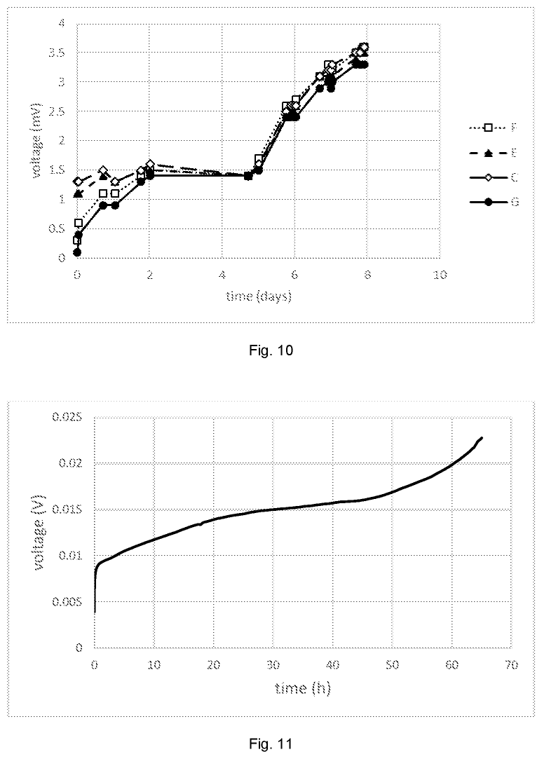

FIG. 10 illustrates a voltage increase during long time current bias (200 nA) for electrode samples having an electrode adhesive according to the present invention (Example 1). Due to the long measurement time the voltage here was not continuously logged but measured only a few times a day (with breaks for weekend). The samples F, E, C, G correspond to nominally identical samples which were current biased while being series connected. Therefore, the results were as expected very similar. An initial variation (DC offset) vanished after two day leading to stable plateau. After about 5 days the voltage started to increase. However, that the voltage was still well below the required limit of 100 mV. Therefore, this test was clearly passed for the 8 days measured (and would be most likely also be passed for longer times).

FIG. 11 illustrates a voltage increase during long time current bias (2 μA) for an electrode sample having an electrode adhesive (Example 1). 2 μA corresponds to ten times the current required by the norm. This test aims at qualifying an accelerated test. The results are roughly corresponding with an increase occurring from 40-45 h. With factoring in the higher current (and figuring that the relevant value is the flown charge) that would correspond to 6 days in the normal test (where 5 days were seen). The voltages here were higher due to Ohm's law (and therefore the beginning of the increase might be hidden).

ANSI/AAMI EC12:2000/(R)2015 requires a peak-to-peak voltage of less than 150 pV (after 1 min stabilization) to guarantee a low noise ECG signal. The AC signal of an electrode sample with electrode adhesive recorded via an ECG system usually has a peak to peak voltage below 10V.

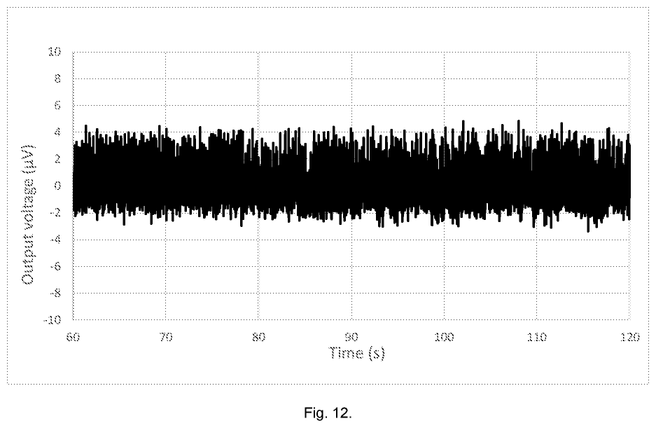

FIG. 12 illustrates an offset instability and internal noise for an electrode sample having an electrode adhesive according to the present invention (Example 1). The measurement corresponds to an ECG measurement with interconnected electrodes instead of a human body. The total bandwidth is about 8V and therefore much lower than required in the norm (150V).

1. An electrode, comprising:
(A) a conductive pressure sensitive adhesive layer, which comprises components:
(A1) at least one (meth)acrylic polymer, which is obtained by polymerizing (meth)acrylic monomers, optionally with vinyl monomers, wherein at least 10 wt.-% of the (meth)acrylic monomers contain at least one —OH group, wt.-% being based on total weight of the (meth)acrylic polymer;
(A2) at least one ionic liquid;
(A3) optionally at least one ionic conductivity promoter different from (A2) and (A4);
(A4) optionally at least one electrically conductive particle;
(A5) optionally at least one polyol; and
(A6) optionally at least one solvent;
(B) a conductive layer, which is in contact with the conductive pressure sensitive adhesive layer (A);
(C) optionally a substrate, which is in contact with the conductive layer (B); and
(D) optionally a release liner, which is in contact with the conductive pressure sensitive adhesive layer (A).

2. The electrode according to claim 1, wherein the (meth)acrylic monomers polymerized to form the at least one (meth)acrylic polymer (A1) are selected from methyl (meth)acrylate, hydroxyethyl acrylate, hydroxypropyl acrylate, hydroxybutyl acrylate, butyl acrylate, ethylhexylacrylate, acrylic acid, C2-C18 alkyl (meth)acrylate, (meth)acrylamide; cyclohexyl (meth)acrylate, glycidyl (meth)acrylate, benzyl (meth)acrylate and combinations thereof, and the (meth)acrylic monomers containing at least one —OH group are present in an amount of at least 15 wt.-% and at most 65 wt.-%, based on the total weight of the acrylic polymer.

3. The electrode according to claim 1, wherein the (meth)acrylic monomers polymerized to form the at least one (meth)acrylic polymer (A1) comprise hydroxyethyl acrylate and at least one of methyl (meth)acrylate, butyl acrylate, and ethylhexylacrylate; wherein the vinyl monomers, if present are selected from vinyl acetate, N-vinyl caprolactam, acrylonitrile, vinyl ether and combinations thereof.

4. The electrode according to claim 1, wherein the at least one ionic liquid (A2) comprises at least one of an imidazolium salt, a choline salt, a pyridinium salt, a pyrrolidinium salt, a phosphonium salt, a sulfonium salt and optionally an ammonium salt; based on total weight of the conductive pressure sensitive adhesive layer.

5. The electrode according to claim 1, wherein the at least one ionic liquid (A2) is selected from the group consisting of imidazolium acetates, imidazolium sulfonates, imidazolium chlorides, imidazolium sulphates, imidazolium phosphates, imidazolium thiocyanates, imidazolium dicyanamides, imidazolium benzoates, imidazolium triflates, choline triflates, choline saccharinate, choline sulfamates, pyridinium acetates, pyridinium sulfonates, pyridinium chlorides, pyridinium sulphate, pyridinium phosphates, pyridinium thiocyanates, pyridinium dicyanamides, pyridinium benzoates, pyridinium triflates, pyrrolidinium acetates, pyrrolidinium sulfonates, pyrrolidinium chlorides, pyrrolidinium sulphates, pyrrolidinium phosphates, pyrrolidinium thiocyanates, pyrrolidinium dicyanamides, pyrrolidinium benzoates, pyrrolidinium triflates, phosphonium acetates, phosphonium sulfonates, phosphonium chlorides, phosphonium sulphates, phosphonium phosphates, phosphonium thiocyanates, phosphonium dicyanamides, phosphonium benzoates, phosphonium triflates, sulfonium acetates, sulfonium sulfonates, sulfonium chlorides, sulfonium sulphates, sulfonium phosphates, sulfonium thiocyanates, sulfonium dicyanamides, sulfonium benzoates, sulfonium triflates, ammonium acetates, ammonium sulfonates, ammonium chlorides, ammonium sulphates, ammonium phosphates, ammonium thiocyanates, ammonium dicyanamides, ammonium benzoates, ammonium triflates and mixtures thereof.

6. The electrode according to claim 1, wherein the at least one ionic liquid (A2) is selected from the group consisting of 1-ethyl-3-methylimidazolium acetate, 1-ethyl-3-methylimidazolium methanesulfonate, 1-ethyl-3-methylimidazolium trifluoromethanesulfonate, 1-ethyl-3-methylimidazolium chloride, 1-ethyl-3-methylimidazolium ethyl sulphate, 1-ethyl-3-methylimidazolium diethyl phosphate, 1-ethyl-3-methylimidazolium thiocyanate, 1-ethyl-3-methylimidazolium dicyanamide, 1-ethyl-3-methylimidazolium benzoate, choline trifluoromethane sulfonate, choline saccharinate, choline acesulfamate, choline N-cyclohexylsulfamate, tris(2-hydroxyethyl)methylammonium methylsulphate, 1-ethyl-3-methylimidazolium tetrafluoroborate, 1-allyl-3-methylimidazolium bis(trifluoromethylsulfonyl)imide, 1-ethyl-3-methylimidazolium ethyl sulphate, choline acetate and mixtures thereof.

7. The electrode according to claim 1, wherein the at least one ionic liquid (A2) is selected from the group consisting of 1-ethyl-3-methylimidazolium benzoate, 1-ethyl-3-methylimidazolium tetrafluoroborate, 1-ethyl-3-methylimidazolium methanesulfonate, 1-ethyl-3-methylimidazolium chloride, 1-ethyl-3-methylimidazolium trifluoromethanesulfonate, choline trifluoromethanesulfonate, 1-ethyl-3-methylimidazolium acetate, choline acetate, 1-ethyl-3-methylimidazolium diethyl phosphate, 1-allyl-3-methylimidazolium bis(trifluoromethylsulfonyl)imide, 1-ethyl-3-methylimidazolium ethyl sulphate, 1-ethyl-3-methylimidazolium thiocyanate, 1-ethyl-3-methylimidazolium dicyanamide, choline saccharinate, choline acesulfamate, 1-ethyl-3-methylimidazolium ethyl sulphate and mixture thereof.

8. The electrode according to claim 1, wherein the at least one (meth)acrylic polymer (A1) is present in an amount of 10 to 99 wt.-% and the ionic liquid (A2) is present in an amount of 0.5 to 50 wt.-%, based on total weight of the conductive pressure sensitive adhesive layer (A).

9. The electrode according to claim 1, wherein the at least one ionic conductivity promoter (A3), different from (A2) and (A4), is present in an amount of 0.1 to 30 wt.-%, based on total weight of the conductive pressure sensitive adhesive layer; and is selected from choline chloride, choline bitartrate, choline dihydrogen citrate, choline phosphate, choline gluconate, choline fumarate, choline carbonate, choline pyrophosphate and mixture thereof.

10. The electrode according to claim 1, wherein the at least one electrically conductive particle (A4) is present in an amount of 0.1 to 35% by weight of total weight of the composition and is selected from the group consisting of metal (nano)particles, graphite (nano)particles, carbon (nano)particles, carbon nanowires, conductive polymer (nano)particles, and mixtures thereof.

11. The electrode according to claim 1, wherein the at least one polyol is present in an amount of 0.1 to 50 wt.-%, based on total weight of the conductive pressure sensitive adhesive layer; and is selected from polyether polyols and mixtures thereof.

12. The electrode according to claim 1, wherein the solvent is present in an amount of 0.001 to 5 wt.-%, based on the total weight of the conductive pressure sensitive adhesive layer (A).

13. The electrode according to claim 1, wherein the conductive pressure sensitive adhesive layer (A) has:
(i) a thickness of 1 to 200 μm; and
(ii) an impedance value of 10¹ to 10⁷Ω at 10 Hz.

14. The electrode according to claim 1, wherein the conductive layer (B) has a thickness of 0.1 to 500 μm; and is selected from a metal layer, a metal salt layer, a carbon layer and mixtures thereof.

15. The electrode according to claim 4, wherein the conductive layer (B) comprises Ag/AgCl and is the only conductive layer contained in the electrode in addition to the conductive pressure sensitive adhesive layer.

16. The electrode according to claim 1, wherein the substrate (C) is present in a thickness of 10 to 500 μm and is a flexible film selected from polyolefin films, polycarbonate films, thermoplastic polyurethane films, silicone films, woven films, non-woven films, and paper films.

17. The electrode according to claim 1, wherein the electrode does not contain more than 0.5 wt.-% of a hydrogel, based on total weight of the electrode; and does not contain more than 0.5 wt.-% of an aqueous electrolyte paste, based on the total weight of the electrode.

18. A method of manufacturing an electrode according to claim 1, comprising steps of:
(i) providing the conductive layer, optionally deposited on one side of the substrate via flat-bed screen printing, rotary screen printing, flexo-printing, gravure printing, pad printing, inkjet printing, LIFT printing, vacuum based deposition methods, spray coating, dip coating or plating;
(ii) applying the conductive pressure sensitive adhesive layer upon the conductive layer, optionally via coating, laminating, spraying or printing; and
(iii) optionally applying the release liner upon a side of the conductive pressure sensitive adhesive layer opposite the conductive layer.

19. The method of claim 18, wherein in step (ii) the conductive pressure sensitive adhesive layer partially or fully covers a surface of the conductive layer.

20. The method of claim 18, wherein after applying the conductive pressure sensitive adhesive layer curing the conductive pressure sensitive adhesive layer for is to 2 h at temperatures of 20 to 150° C.

21. The method of claim 18, wherein after the application of the conductive layer, the conductive layer is dried for 1 second to 2 hours at 20 to 200° C.

22. A method of monitoring biosignals comprising steps of: obtaining the electrode according to claim 1; after removal of the release liner, if present, from the electrode, applying the conductive pressure sensitive adhesive layer of the electrode to skin such that the electrode is removably adhered to the skin; and monitoring biosignals from the skin via the electrode.
